(19) United States
(12) Patent Application Publication
(10) Pub. No.: US 2017/0014805 A1
(43) Pub. Date: Jan. 19, 2017

(54) METHOD FOR THE SYNTHESIS OF SUPPORTED GOLD (AU) NANOPARTICLES FOR EPOXIDATION REACTIONS

(71) Applicant: SABIC GLOBAL TECHNOLOGIES B.V., Bergen op Zoom (NL)

(72) Inventor: Mohammed H. AL-HAZMI, Riyadh (SA)

(21) Appl. No.: 15/301,110

(22) PCT Filed: Apr. 21, 2015
(86) PCT No.: PCT/IB2015/052917

§ 371 Date: Sep. 30, 2016

Related U.S. Application Data
(60) Provisional application No. 61/982,498, filed on Apr. 22, 2014.

(51) Int. Cl. B01J 23/52; B01J 21/10; B01J 21/04; B01J 23/06; C07D 301/10; B01J 23/755; B01J 37/02; B01J 37/08; B01J 35/00; B01J 35/02; B01J 21/06; B01J 23/745

(52) U.S. Cl. CPC B01J 23/52; B01J 21/063; B01J 21/10; B01J 21/04; B01J 23/06; B01J 23/745; B01J 23/755; B01J 37/0219; B01J 37/0236; B01J 37/08; B01J 35/0013; B01J 35/023; C07D 301/10

(57) ABSTRACT

Processes for preparing supported gold nanoparticle catalysts are provided. In an exemplary embodiment, the process includes adding a solution of a phosphorus compound to a solution of chloro (dimethyl sulfide) gold (I) to obtain a solution of chloro (phosphorus compound) gold (I) complex, adding the solution of chloro (phosphorus compound) gold (I) complex to a solution of silver nitrate to obtain a solution of nitro (phosphorus compound) gold (I) complex, applying the solution of nitro (phosphorus compound) gold (I) complex to a metal hydroxide support, drying the metal hydroxide support; and calcining the dried metal hydroxide support to form the supported gold nanoparticle catalyst. Supported gold nanoparticle catalysts prepared by the process and processes for oxidizing ethylene to ethylene oxide in the presence of the supported gold nanoparticle catalysts are also provided.

### FIELD

The presently disclosed subject matter relates to processes for preparing supported gold nanoparticle catalysts, supported gold nanoparticle catalysts prepared thereby, and processes for oxidizing ethylene to ethylene oxide in the presence of the supported gold nanoparticle catalysts.

### BACKGROUND

Ethylene oxide is an important chemical intermediate in many industrial processes for manufacturing many products used in a wide range of downstream markets. Ethylene oxide is a colorless gas at room temperature and condenses to liquid at 10° C. Ethylene oxide is miscible with water and many other organic solvents, such as alcohols and ethers. Additional physical and chemical properties of ethylene oxide are summarized in Table 1. The reactivity of ethylene oxide is attributed to its highly stained 3-member ring.

TABLE 1
Physical properties of ethylene oxide

| Property | Value |
|---|---|
| Boiling point (° C.) at 101.3 kPa | 10.4 |
| Melting point (° C.) | −112.4 |
| Heat of combustion at 25° C. (kJ/mol) | 1306.04 |
| Heat of Vaporization (cal. g) at 1 atm | 136.1 |
| Density | 0.8711 |
| Decomposition Temperature in air (° C.) | 560 |
| Dipole Moment (c-m) | 634 × 10⁻³⁰ |
| Solubility | Water, Acetone, Benzene |

Ethylene oxide can be converted to ethylene glycol via a non-catalytic hydrolysis reaction. Ethylene glycol serves as a raw material for the production of polymers such as polyethylene terephthalate and can be used as automobile anti-freeze additive. Ethylene oxide is also utilized for the production of ethanolamine, solvents, surfactants, etc. FIG. 1 illustrates various products obtained from ethylene oxide.

Ethylene oxide can also be produced by a chlorohydrin process. In this process, the reaction proceeds via two sequential reactions shown in FIG. 2. First, hypochlorous acid is formed as a result of the reaction of chlorine with water and reacts with ethylene to form ethylene chlorohydrin and hydrochloric acid. Next, an intermolecular displacement of chloride from ethylene chlorohydrin takes place in the presence of a base such as calcium hydroxide (Monison, et al., Organic Chemistry, Fifth Edition, Allyn and Bacon, Inc.: Boston, 1987, p. 713), as shown in FIG. 2.

The chlorohydrin process has been replaced by the direct heterogeneous catalytic oxidation of ethylene to ethylene oxide over supported silver based catalyst. One aspect of this process is the selection of a catalyst system that minimizes complete oxidation of ethylene to CO₂. The major routes to carbon dioxide and water are either by direct oxidation of ethylene or further oxidation of ethylene oxide (Schonfeldt, et al., Surface Active Ethylene Oxide Adducts, Second Edition, Pergamon Press Ltd.: Hungary, 1969, p. 25; Kilty, et al., The Mechanism of the Selective Oxidation of Ethylene to Ethylene Oxide. Catalyst Reviews (1974):10(1):1).

Furthermore, a pathway for converting ethylene oxide to carbon dioxide and water is through its isomerization product acetaldehyde (Weissermel et al.). Minor amounts of formaldehyde are detected in the commercial process as a result of the direct oxidation of ethylene (Raben, F. Ethylene Oxide Manufacturing Process. Reviews in Process Chemistry and Engineering (1999):2(1):53). The selectivity and the conversion rate can be directly affected by the nature of the catalyst and the reaction conditions. FIG. 3 demonstrates the major products from a direct oxidation of ethylene. All the ΔH values in FIG. 3 were calculated using ΔH_{f} values at 298 K (Atkins, P. W. Physical Chemistry, Fifth Edition, Oxford University Press, London (1994):C9-C10).

The silver-oxygen interaction is an important processes involved in ethylene epoxidation. Many studies have been undertaken to elucidate the chemical interaction between silver metal and oxygen, such as adsorption, desorption, effects of surface contamination, and the reactivity of several crystalline silver phases (Czanderna A. W. The Adsorption or Oxygen on Silver, The Journal of Physical Chemistry (1964);68(10), 2765).

The mechanism of selective oxidation of ethylene to ethylene oxide in a gas phase has been disclosed. Molecular oxygen can be chemisorbed either dissociatively to form atomic oxygen or non-dissociatively to yield a surface-bound oxygen molecule. The molecular oxygen subsequently reacts with ethylene to give ethylene oxide, while atomic oxygen reacts to give the total oxidation products. The above noted article by Cazandera suggested a mechanism for adsorption of oxygen on silver metal in three processes as follows. First, a low activation energy,(3 kcal mol⁻¹), dissociative adsorption of oxygen molecule on four adjacent silver atoms.

O₂+4Ag (adj)→2O²⁻ (ids)+4Ag⁺ (adj)

A second process takes place at sites where four adjacent silver atoms are not available. This leads to molecular oxygen adsorption with an activation energy higher than that of the first process (8 kcal mol⁻¹).

O₂+Ag→O₂⁻ (ads)+Ag⁺

The third process is a dissociative adsorption of oxygen on four non-adjacent silver atoms to form adsorbed atomic oxygen and four adjacent silver atoms. This takes places via surface oxygen migration at an elevated temperature and with higher activation energy (14 kcal mol¹⁻).

O₂+4Ag (non-adj)→2O²⁻ (ads)+4Ag⁺ (adj)

Metallic gold was considered to be inactive as a catalyst for a long period of time. The poor activity and the nobility of the macroscopic large gold particles in catalysis was attributed to its low chemisorption affinity and negative enthalpy of chemisorptions of oxygen on gold surface (Hammer, et al., Gold Is the Noblest of All the Metals, Nature (1995);376:238). The latter was the weak overlap between the molecular or atomic orbitals of the incoming molecule or atom with the gold d-orbitals. However, it is now known that gold can exhibit high catalytic activity under certain conditions (Bond, G. Gold: a Relatively New Catalyst, Catalysis Today (2002);72:5). When gold is supported on a surface of a metal oxide as fine nanoparticles, it can show remarkable reactivity and may be advantageous in many catalytic reactions such as water gas shift (Tabakova et al., Influence of the Microscopic Properties of the Support on the Catalytic Activity on Au/ZnO, Au/Fe₂O₃, Au/Fe₂O₃—ZnO, Au/Fe₂O₃—ZrO Catalysts for the WGS Reaction, Applied Catalysis A, 2000, 202,91), hydrochlorination of acetylene (Thomson, New Advances in Gold Catalysis, Part 1. Gold Bulletin (1998);31(4):111), oxidation of carbon monoxide (Grunwaldt et al., Preparation of Supported Gold Catalyst for Low-Temperature CO Oxidation Via Size-Controlled Gold Colloids, Journal of Catalysis (1999);181:223; Haruta et al., Advances in the Catalysis of Au Nanoparticles, Applied Catalysis (2001);222:427), reduction of nitrogen oxides to nitrogen gas (Ueda et al., Nitric Oxide Reduction with Hydrogen, Carbon Monoxide, and Hydrocarbons Over Gold Catalysis, Gold Bulletin (1999);32:1), hydrogenation (Mohr et al., The Influence of Real Structure of Gold Catalyst in the Partial Hydrogenation of Acrolein, Journal of Catalysis (2003);213:86), and selective oxidation (Morrison et al., Thomas, New Advances in Gold Catalysis, Part II, Gold Bulletin (1999);32(1); Mul et al., Stability and Selectivity of Au/TiO2 and Au/TiO₂/SiO₂ Catalysts in Propane Epoxidation: An in Situ FT-IR Study, Journal of Catalysis (2001);201:128; Uphade et al., Vapor-Phase Epoxidation of Propane Using H₂ and O₂ Over Au/Ti-MCM-48, Journal of Catalysis (2002);209:331; WO 2003/062196). Furthermore, activated supported gold nanoparticles have been employed in direct epoxidation of propylene (Grunwaldt et al.; Mul et al.; Uphade et al.; WO 2003/062196).

Richardson et al. studied the adsorption of molecular oxygen on gold films at a wide range of temperatures (25-405° C.) (Richardson et al., The Absorption of Oxygen on Evaporated Gold Films, Journal of Catalysis (1970); 46:420). It was found that no adsorption of oxygen took place in the temperature range of 25-230° C., while low coverage (θ=0.3) occurred at 341° C. and a maximum coverage of (θ=0.7) was realized at 405° C. Pireaux characterized oxygen adsorption over clean gold surfaces using high resolution electron energy loss spectroscopy (HREED), X-ray photoelectron spectroscopy (XPS), and Auger spectroscopy (Pireaux et al., Spectroscopy Characterization of Oxygen Absorption on Gold Surfaces. II. Production of Gold Oxide in Oxygen DC Reactive Sputtering, Surface Science (1984);141:221). It was found that molecular oxygen did not adsorb on Au (110) and Au (111) crystal faces in the temperature range of 100-800 K. However, it has also been suggested that oxygen molecules can adsorb on negatively charged gold clusters (Salisbury et al., Low-Temperature Activation of Molecular Oxygen by Gold Clusters: As Stoichiometric Process Correlation to Electron Affinity. Chemical Physics (2000);262:131). Mills reported the adsorption of molecular oxygen on neutral and charged gold clusters (Mills et al., The Absorption of Molecular Oxygen on Neutral and Negative Au, clusters (n=2-5), Chemical Physics Letters (2002);306:493). Moreover, Franceschetti et al. reported the adsorption of oxygen molecules on gold clusters with a binding energy of 0.5 to 1.5 ev (Franceschetti et al., Oxygen chemisorptions on Au Nanoparticles, Chemical Physics (2003);274:471). Francescheui et al. found that the binding energy of molecularly adsorbed oxygen was smaller than that of dissociative adsorbed atomic oxygen, which includes a nearly liner O—Au—O bridge. Salisbury suggested that oxygen molecule acts as a one electron acceptor and is bonded to gold nanoparticles as a stable superoxide ion O₂⁻ (Salisbury et al.). The stability of the superoxide ion was attributed to the reduction of the number of unpaired electrons in the π* anti-bonding orbitals from two electrons to one (Salisbury et al.).

There remains a need in the art for an improved catalyst for selective oxidation of ethylene to ethylene oxide that reduces the oxidation of ethylene to carbon dioxide.

### SUMMARY

Disclosed herein are processes for preparing a supported gold nanoparticle catalyst. The process includes adding a solution of a phosphorus compound to a solution of chloro (dimethyl sulfide) gold (I) to obtain a solution of chloro (phosphorus compound) gold (I) complex, where the phosphorus compound can be a phosphine having a formula of PR₁R₂R₃, a phosphinite having a formula of P(OR₄)R₅R₆, a phosphonite having a formula of P(OR₇)(OR₈)R₉, a phosphite having a formula of _{P}(OR₁₀)(OR₁₁)(OR₁₂), or a combination thereof. Each of R₁ to R₁₂ can be an alkyl, an optionally substituted alkyl, an aryl, an optionally substituted aryl, an aralkyl, an optionally substituted aralkyl, or a combination thereof. The process further includes adding the solution of chloro (phosphorus compound) gold (I) complex to a solution of silver nitrate to obtain a solution of nitro (phosphorus compound) gold (I) complex, applying the solution of nitro (phosphorus compound) gold (I) complex to a metal hydroxide support, drying the metal hydroxide support; and calcining the dried metal hydroxide support to form the supported gold nanoparticle catalyst. In some embodiments, the alkyl can be i-propyl, cyclohexyl, t-butyl, ethyl, or a combination thereof. In some embodiments, the aryl is phenyl. In some embodiments, the optionally substituted aryl is mesityl, 2,6-dimethoxyphenyl, 4-methoxyphenyl, 1-naphthyl, 4-methylphenyl, or a combination thereof. In some embodiments, the aralkyl is benzyl. In certain non-limiting embodiments, the phosphorus compound is a phosphine having a formula of PR₁R₂R₃

In non-limiting embodiments, the metal hydroxide is aluminum hydroxide, magnesium hydroxide, zinc hydroxide, iron hydroxide, nickel hydroxide, titanium hydroxide, or combinations thereof. In some embodiments, the metal hydroxide is obtained by hydrolysis of the metal in a solution of potassium hydroxide. In one embodiment, the metal hydroxide is titanium hydroxide, and the titanium hydroxide is obtained by hydrolysis of titanium-tetra-isopropoxide in a solution of potassium hydroxide. In certain embodiments, the hydrolysis is carried out in a pH value of from about 5 to about 10. The pH value can be adjusted by addition of the potassium hydroxide solution.

In certain embodiments, the solution of nitro (phosphorus compound) gold (I) complex is applied to the metal hydroxide with a continuous stirring for about 12 hours. The metal hydroxide is dried under vacuum at a temperate of from about 20° C. to about 25° C. The dried metal hydroxide is calcined at a temperate of from about 100° C. to about 300° C. In some embodiments, the process is carried out in an inert nitrogen atmosphere. Additionally, the process can further include adding an alkali promoter to the solution of nitro (phosphorus compound) gold (I) complex before applying the solution of nitro (phosphorus compound) gold (I) complex to the metal hydroxide support. In one embodiment, the alkali promoter is cesium.

The presently disclosed subject matter also provides supported gold nanoparticle catalysts prepared by the above-described processes. In some embodiments, the size of the gold nanoparticle is from about 2 nm to about 15 nm. In non-limiting embodiments of the presently disclosed subject matter, the supported gold nanoparticle catalyst includes from about 0.1% to about 5% by weight of the nitro (tri-alkyl-phosphine) gold (I) complex.

Additionally, the presently disclosed subject matter provides processes for oxidizing ethylene to ethylene oxide. The processes include reacting ethylene and oxygen in the presence of the above-described supported gold nanoparticle catalysts. In some embodiments, the process is carried out in a fixed bed flow reactor. In certain embodiments, an inert gas is fed to the oxidization process. In one embodiment, the inert gas is argon.

### BRIEF DESCRIPTION OF THE DRAWINGS

FIG. 1 represents products obtained from ethylene oxide.

FIG. 2 illustrates synthesis of ethylene oxide via a chlorohydrin process.

FIG. 3 illustrates direct oxidation of ethylene.

FIG. 4 shows a process for synthesizing a supported gold nanoparticle catalyst in accordance with one non-limiting exemplary embodiment of the presently disclosed subject matter.

FIG. 5 represents a reactor system for oxidizing ethylene to ethylene oxide in the presence of a supported gold nanoparticle catalyst prepared by the process in accordance with one non-limiting exemplary embodiment of the presently disclosed subject matter.

FIG. 6 represents one mechanism for selective oxidation of ethylene to ethylene oxide over the supported gold nanoparticle catalyst prepared by the process in accordance with one non-limiting exemplary embodiment of the presently disclosed subject matter.

FIG. 7 represents one mechanism for selective oxidation of ethylene to ethylene oxide over the supported gold nanoparticle catalyst prepared by the process in accordance with one non-limiting exemplary embodiment of the presently disclosed subject matter.

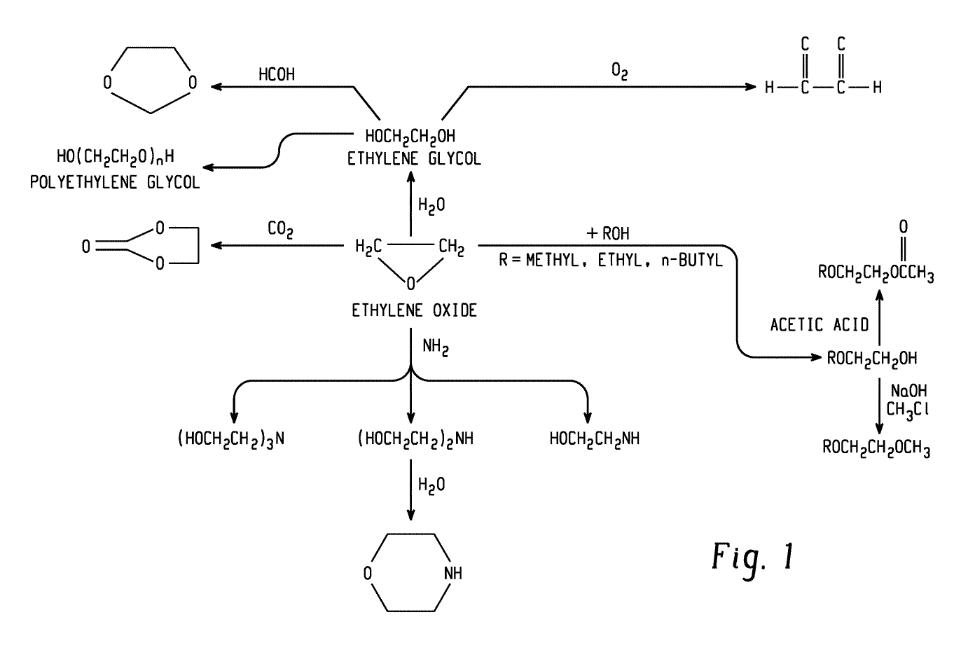

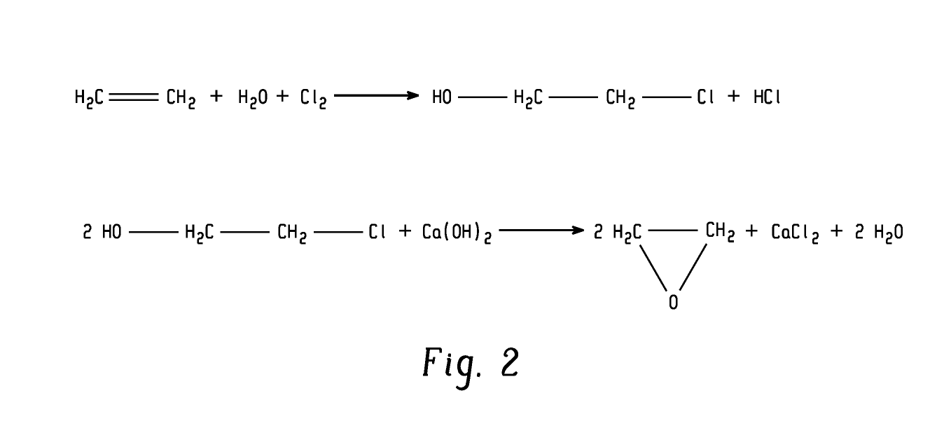

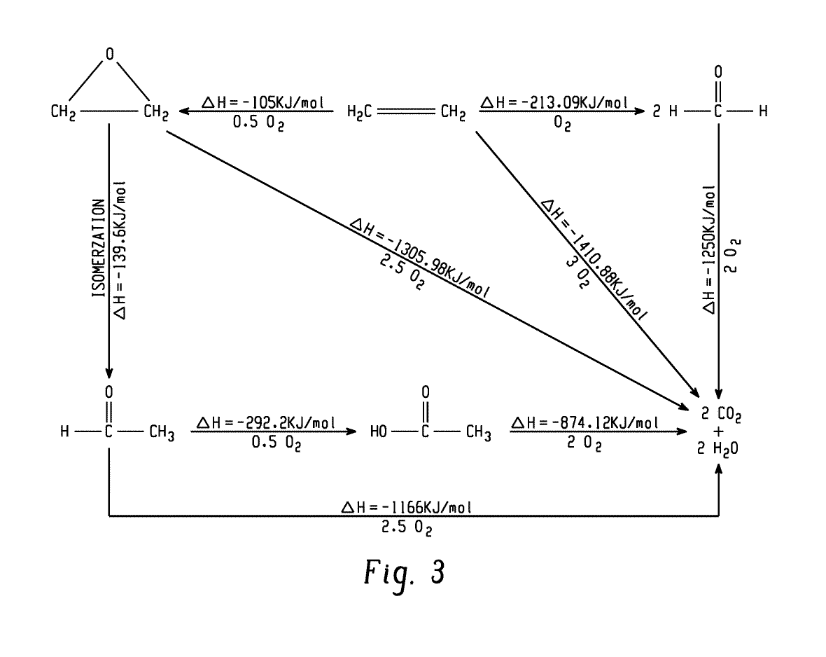

### DETAILED DESCRIPTION

The presently disclosed subject matter provides processes for preparing supported gold nanoparticle catalysts, supported gold nanoparticle catalysts prepared thereby, and processes for oxidizing ethylene to ethylene oxide in the presence of the supported gold nanoparticle catalysts.

Synthesis of Supported Gold Nanoparticle Catalysts

According to the presently disclosed subject matter, the synthesis processes are carried out in an inert nitrogen atmosphere. The processes can take place inside a glove box with low light conditions. An exemplary process of synthesizing the supported nanoparticle gold catalysts in accordance with the presently disclosed subject matter can include : (i) synthesis of phosphorous compound ligands; (ii) synthesis of chloro (phosphorus compound) gold (I) complexes (e.g., chloro (alkyl-phosphine) gold (I) complexes (R₁R₂R₃PAuCl)); (iii) synthesis of nitro (phosphorous compound) gold (I) complexes (e.g., nitro (alkyl phosphine) gold (I) complexes), and (iv) synthesis of supported gold nanoparticles. Each is now described in turn.

1. Synthesis of phosphorous compound Ligands

Phosphorous compound ligands can be obtained from various commercial sources. For example, all tertiary phosphines except trinaphthyl phosphine can be acquired from the Aldrich Company. Trinaphthyl phosphine can be purchased from Alfa Company.

2. Synthesis of Chloro (phosphorous compound) Gold (I) Complexes

Chloro (phosphorous compound) gold (I) complexes can be synthesized by adding a solution of phosphorus compound to a solution of chloro (dimethyl sulfide) gold (I). In certain embodiments, the phosphorus compound is a phosphine having a formula of PR₁R₂R₃. Each of R₁, R₂, and R₃ can be an alkyl, an optionally substituted alkyl, an aryl, an optionally substituted aryl, an aralkyl, or an optionally substituted aralkyl. R₁, R₂, and R₃ can be the same or different from each other.

In certain embodiments, the phosphorus compound is a phosphinite having a formula of P(OR₄)R₅R₆. Each of R₄, R₅, and R₆ can be an alkyl, an optionally substituted alkyl, an aryl, an optionally substituted aryl, an aralkyl, or an optionally substituted aralkyl. R₄, R₅, and R₆ can be the same or different from each other.

In certain embodiments, the phosphorus compound is a phosphonite having a formula of P(OR₇)(OR₈)R₉. Each of R₇, R₈, and R₉ can be an alkyl, an optionally substituted alkyl, an aryl, an optionally substituted aryl, an aralkyl, or an optionally substituted aralkyl. R₇, R₈, and R₉ can be the same or different from each other.

In certain embodiments, the phosphorus compound a phosphite having a formula of P(OR₁₀)(OR₁₁)(OR₁₂). Each of R₁₀, R₁₁, and R₁₂ can be an alkyl, an optionally substituted alkyl, an aryl, an optionally substituted aryl, an aralkyl, or an optionally substituted aralkyl. R₁₀, R₁₁, and R₁₂ can be the same or different from each other.

In certain embodiments, the alkyl is i-propyl, cyclohexyl, t-butyl, or ethyl. In certain embodiments, the aryl is phenyl. In certain embodiments, the optionally substituted aryl is mesityl, 2,6-dimethoxyphenyl, 4-methoxyphenyl, 1-naphthyl, or 4-methylphenyl. In certain embodiments, the aralkyl is benzyl.

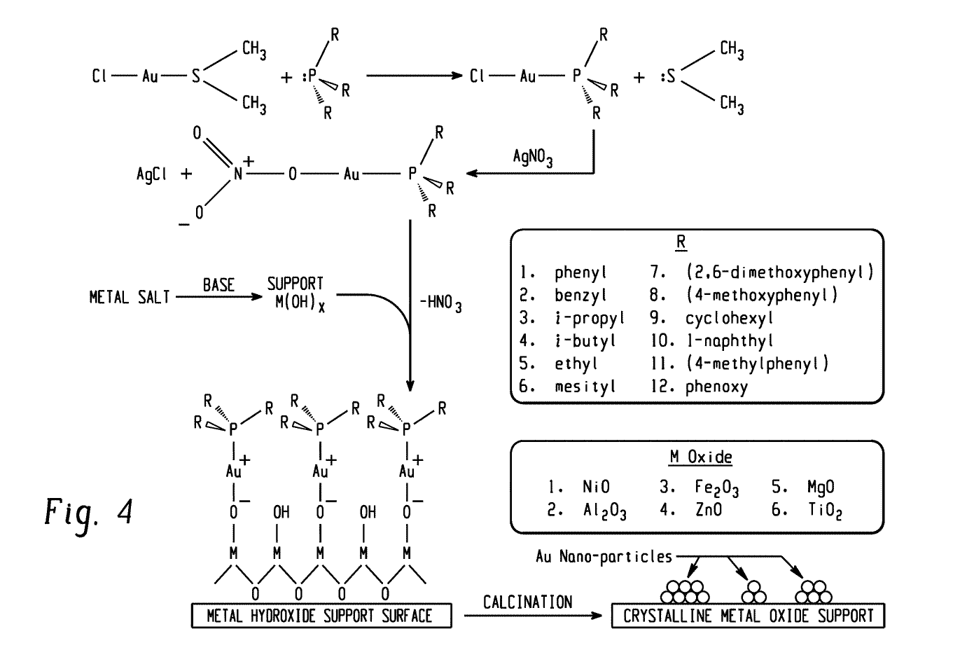

In one non-limiting example, as shown in FIG. 4, a solution of tri-alkyl-phosphine in dichloromethane can be added drop-wise to a solution of chloro (dimethyl sulfide) gold (I) in dichloromethane with an equal molar ratio of phosphine ligands to gold complex. Chloro (tri-alkyl-phosphine) Gold (I) (R₃PAuCl) complex is precipitated by a slow addition of n-hexane to the reaction mixture. The product can then be filtered off to remove all the residual dimethyl sulfide and dichloromethane. Subsequently, the product is re-crystallized from a mixture of n-hexane and dichloromethane (Bruce et al., Synthesis of Gold-Containing Mixed-Metal Cluster Complexes, Inorganic Synthesis (1989);26:324).

3. Synthesis of Nitro (phosphorus compound) Gold (I) Complexes

In one non-limiting example, as shown in FIG. 4, a solution of the chloro (tri-alkyl-phosphine) gold (I) complex in dichloromethane can be added to a solution of silver nitrate in methanol. A white precipitate of silver chloride is formed immediately. The mixture can then be stirred, e.g., for about one hour at room temperature, and the solution filtered to remove the silver chloride powder (Mueting et al., Mixed-Metal-Gold Phosphine Cluster Compounds, Inorganic Synthesis (1992);29:279). The filtrate can be left for crystallization by slow solvent removal. The product is re-crystallized from a mixture of dichloromethane and n-hexane.

### 4. Synthesis of Supported Gold Nanoparticles

The metal hydroxide support can be aluminum hydroxide, magnesium hydroxide, zinc hydroxide, iron hydroxide, nickel hydroxide, titanium hydroxide, or combinations thereof. As is understood by those of ordinary skill in the art, the term “metal hydroxide” can included various oxides and hydrates of the metal. The metal hydroxide support can be obtained by hydrolysis of the metal, e.g., aluminum, magnesium, zinc, iron, and nickel nitrates, in an aqueous solution of potassium hydroxide. In one embodiment, the metal hydroxide is titanium hydroxide. Titanium hydroxide can be obtained by hydrolysis of titanium-tetra-isopropoxide in a solution of potassium hydroxide. The pH value can be adjusted by gradual addition of the potassium hydroxide solution. The metal hydroxide precipitate can be filtered off and washed repeatedly by distilled water.

In one non-limiting example, as shown in FIG. 4, an acetone solution of the gold nitrate complex obtained as described above 3 can be impregnated into the wet metal hydroxide with continuous stirring for about 12 hours, e.g., in a similar manner as described for analogous compound by Kozlov (Kozlov et al., Supported Gold Catalysts Prepared from a Gold Phosphine Precursor and As-Precipitated Metal-Hydroxide Precursor: Effect of Preparation Conditions on the Catalytic Performance, Journal of Catalysis (2000);196:56). The theoretical metallic gold concentration in the impregnation solution can be altered by variation of the amount of gold complex introduced. The final solution can be dried under vacuum at a room temperature (20° C. to about 25° C.) followed by a calcining process with a ramping rate of 2 K/min to different temperatures in the range of 100° C. to about 300° C.

Phosphine atoms can make gold more cationic allowing it to interact more strongly with the support hydroxyl groups leading to the formation of very small particles upon thermal treatment. More cationic supported gold nanoparticles can exhibit superior catalytic performance for selective oxidation of ethylene to ethylene oxide. .

The presently disclosed subject matter also provides supported gold nanoparticles prepared by the above-described processes. Three factors that can be adjusted in a supported gold nanoparticle catalyst in order to achieve superior catalytic performance. The first is the nature of the support and the strong interaction of the gold nanoparticles with the surface of the support to form deposited hemispherical nanoparticles. The support is important for the provision of surface anion vacancies in which oxygen can be adsorbed. Additionally, a porous structure in the support provides a high surface area. In certain embodiments, the supported gold nanoparticle catalyst of the presently disclosed subject matter includes aluminum hydroxide, Al₂O₃ as a neutral support, iron hydroxide, Fe₂O₃ as a reducible metal oxide support, nickel hydroxide, NiO as an oxidizable support, magnesium hydroxide, zinc, hydroxide, MgO and ZnO as basic supports, titanium hydroxide, and TiO₂ as an acidic support.

The second factor is the gold particle size, as maintaining ultra-fine gold nanoparticles with particle size of from about 0.5 nm to about 15 nm (e.g., from about 0.5 nm to about 5 nm, or from about 2 nm to about 15 nm) is important for the catalytic properties. In one embodiment, the gold particle size is from about 2 nm to about 15 nm. When the size of the gold particles decreases, each atom behaves more as individual atoms as a result of a weak bond structure, which can make the gold metal less noble. However, very small supported gold nanoparticles (=0.8 nm) have shown very little catalytic activity for CO oxidation reaction due to a low surface fraction available for chemical interaction (Salisbury et al.; Sanchez et al., When Gold is Not Noble: Nanoscale Gold Catalysts, Journal of Physical Chemistry A (1999);103:9573).

The third factor is high dispersion of gold nanoparticles on the surface of the support, which can be important for high catalytic activity of a supported gold catalyst (Haruta, Catalysis of Gold Nanoparticles Deposited on Metal Oxides. Cattech (2002);6(2):102). All of the three factors can be affected by the preparation method of the supported gold nanoparticles.

The supported gold nanoparticle catalysts of the presently disclosed subject matter can be synthesized by supporting tertiary alkyl-phosphine gold (I) nitrate complexes over a metal hydroxide support. By variation of the alkyl groups attached to the phosphine atoms, the electronic and steric properties of the complexes are varied in order to influence the particle size, dispersion of nanoparticles over the support, and interaction with the support. Increasing electron-negativity of the alkyl group leads to stabilization and lowering energy of the σ* orbitals of the phosphine. Therefore, the empty σ* orbitals on the phosphine is more accessible for the back donation of electrons from the metal orbitals, which makes the gold more cationic that allows the gold to interact more strongly with the support hydroxyl groups, which leads to the formation of very small particles upon thermal treatment.

The steric size of the alkyl groups can also play a role in the dispersion of gold nanoparticles over the support. When one of the gold precursors deposits on the surface of the support, the size of the ligands can restrict access to neighboring hydroxyl groups of the support, and thus, controls the deposition size and nanosize distribution of gold particles. Furthermore, variation of the ligands influences the closed-shell Au—Au aurophilic attraction in the solid state and can have a direct affect on the particle size and dispersion. Electronegative or bulky groups can decrease the Au—Au interaction energy resulting in formation of monomer or dimer molecules (Toronto et al., Solid State Structures and Gold-Gold Bonding in Luminescent Halo(dimethylphenylphosphine)gold (I) Complexes, Inorganic chemistry (1996);35:2484). On the other hand, less electronegative or small ligands can lead to an increase in the aurophilic interaction energy and formation of a polymeric chain of molecules (Mathiesona et al., Preparation and Structural Characterization of Isocyanide Gold (I) Nitrates, [Au(NO₃)(CNR)] (R═Et, BU¹ or C₆H₃Me₂-2,6); New Aurophilic Motifs, Journal of Chemical Society, Dalton Transaction (1999);2:201).

### Characterization of Gold Precursors and Supported Gold Catalyst

Infrared (IR) spectra was collected for the synthesized tri-alkyl-phosphine gold chloride and nitrate complexes. New strong bands appear at 1499 cm⁻¹ and 1275 cm⁻¹ that are characteristic of the NO³⁻ ligands when the chlorides are converted to nitrates. Additionally, the C—H stretching frequencies of the ligands attached to the phosphorous can be observed using IR analysis. IR can also be used to monitor the dissociative absorption of the gold precursor on the support due to interaction with the hydroxyl groups. This can be achieved by observing the disappearance of the NO³⁻ bands and the formation of a new band near 1363 cm⁻¹ that corresponds to the ionic NO³⁻ species on the support (Yuan et al., Supported Au Catalysts Prepared from Au Phosphine Complexes and As-Precipitated Metal Hydroxides: characterization and Low-Temperature CO Oxidation, Journal of Catalysis (1997);1780:191; Yuan et al., Supported Gold Catalysis Derived from the Interaction of a Au-Phosphine Complex with As-Precipitated Titanium Hydroxide and Titanium Oxide, Catalysis Today (1998);44:333). Elemental analysis can be employed to determine the chemical composition of the gold complexes. Metallic gold and phosphorus can be analyzed by X-ray fluorescence spectroscopy. Carbon and nitrogen can be analyzed by CHN microanalysis. Thermogravimetric analysis (TGA) can be used to determine the decomposition temperature of the complexes and catalyst precursors and to identify appropriate temperatures for catalyst synthesis.

The surface area of exposed metallic gold is proportional to the actual catalytic area (Satterfield, Heterogeneous Catalysis in Industrial Practice, Second Edition, McGraw-Hill: New York, 1996: 139). The temperature programmed desorption (TPD) technique can be utilized for quantitative measurement of the surface area of the gold nanoparticles by measuring the uptake of carbon monoxide gas. The TPD technique can also be utilized for studying the surface oxygen coverage effects on the selectivity and activity. This can be performed by measuring the binding strength of oxygen on the surface at different coverage percentages (Czanderna, Isosteric Heat of Absorption of Oxygen on Silver, Journal of Vacuum Science Technology (1977);14:408). The TPD experiments can be performed in a U-type quartz reactor tube of about 6 mm i.d. A sample of 3 g of the catalyst can be placed in the cell and can be healed to 800 K in a continuous flow of 30 cc/min of helium inert gas for surface degassing. Then, the cell can be cooled down to room temperature in a helium atmosphere, and the flow can be switched to the probe gas (O₂ or CO₂) in order to allow it to be adsorbed on the surface for about 30 minutes at room temperate. The flow of helium can be introduced again for about 30 minutes at a flow rate of 30 cc/min. Temperature programming can then be initiated and the TPD spectra can be obtained at temperatures between 273 and 800K with a heating rate of 1 K/s.

High resolution transmission electron microscopy (TEM) can be used for studying the arrangement and size distribution of metallic gold particles on the surface of the catalysts. TEM can be used to calculate the average particle size d using the following formula d=Σnᵢdᵢ/Σnᵢ, where nᵢ is the number of particles of diameter dᵢ.(Kozlov et al.). Approximately, 300 particles are chosen in order to determine the average diameter of gold particles. TEM can be recorded for the as-synthesized as well as the used catalysts to study the effect of reaction conditions on the distribution and particle size of the metallic gold. Surface area is important as heterogeneous catalytic reactions occur at the surface of the solid catalyst, and the rate of the product formation is a function of the surface area of the supported catalyst. The total surface area of the catalysts can be obtained using conventional Brunauer-Emmett-Teller (BET) multilayer nitrogen adsorption methods. Solid state ³¹P nuclear magnetic resonance NMR is one of the most valuable spectroscopic techniques for characterization of phosphine complexes. The ³¹P chemical shift and the ¹J Au-P coupling constant can be influenced by the cone angle and the electro-negativity of the ligands coordinated to the phosphine atom (Silva et al., Vibrational and Solid State (CP/MAS) ³¹P NMR Spectroscopic Studies of Bis(trimethylphosphine) Gold (I) Halides, Journal of Molecular Structure (2000);516:263). The ³¹P NMR spectra can be recorded for the individual tri-alkyl-phosphine ligands, for the gold (I) nitrate precursor complexes, and for the supported gold complexes, which facilitates the study of the variation of the chemical shift due to chemical interaction of gold complex with the support surface. Moreover, it is worth recording the ³¹P NMR chemical shifts for the PR₃ ligands adsorbed on the support and compare them with those of supported metal complexes, which assists the determination of the mechanism of the decomposition of the complex on the support. The ³¹P NMR spectra can be obtained on a 400 MHz NMR spectrometer by solid state cross-polarization spinning at room temperature. The ³¹P coordination chemical shift (Δδ=δ complex−δ ligands) can be measured relative to an external reference of 85% H₃PO₄.

X-ray Photoelectron Spectroscopy (XPS) can be used to measure the binding energies of the Au 4f, P 2p, and O 1 s orbitals of the gold precursor, and the supported gold precursor before and after thermal calcination. Monitoring of the position of the Au 4 f binding energy can give a clear indication about the formal oxidation state of gold. The value of the binding energy can reflect the chemical nature of gold on the surface of the support. The 4f binding energy around 84.4 ev corresponds to metallic gold while that between 86.5 ev and 88.3 ev are attributed to ionic gold Au (Czanderna; Lin et al., Gold Supported on Surface Acidity Modified Y-Type and Iron/Y-type Zeolite for CO Oxidation, Applied Catalysis B (2002);36:19). A 4 f binding energy of gold on the surface of less than 84 ev indicates the formation of large gold particles on the support surface. The X-ray powder diffraction technique (XRD) can be utilized for monitoring the formation of metallic gold particles on the support after thermal calcinations. This can be achieved by observation of the Au (200) peak at 2 θ=44.4° and Au (111) peak at 2 θ=38.2° (Yuan et al., (1997). The sharpness and intensity of the Au (111) diffraction peak gives an indication about the size of the gold particle on the surface of the support. The more sharp and intense the peak, the bigger the gold crystallite size. XRD patterns can be recorded using an X-ray diffractometer with Cu Kᵤ radiation over the 2 θ range of 10°-80°.

Effects can be made to grow single crystals of tertiary tri-alkyl-phosphine gold (I) nitrate complexes suitable for X-ray analysis. Determination of the crystal structure can be useful to evaluate the metal-metal closed-shell intermolecular interaction (Mathieson et al., The Solid State Aggregation of Two Gold (I) Nitrate Complexes, Journal of Chemical Society, Dalton Transaction (2000); 3881). This interaction is strongly dependent on the nature of the alkyl ligands coordinated to the phosphine atoms, such as electro-negativity and steric effects. This kind of interaction can be directly correlated with the dispersion and particle size of the metallic gold formed upon pyrolysis of the supported gold complexes. Atomic force microscopy (AFM) can be used to investigate surface morphology before and after deposition of the gold complex on the surface of the support. Moreover, the effect of the calcination temperature can be inspected by collection of the topographic images of the samples at different calcination temperatures. This kind of analysis can give an impression about the susceptibility of the metal particles toward agglomeration (Ken-ichi et al., Atomic Force Microscopy Study on Thermal and UV-Irradiative Formation and Control of Au Nana-particles on TiO₂(110) From Au(PPh3)(NO₃), Journal of Physical Chemical Physics (2001);3:3871). The particle size distribution and the height distribution can be calculated at each calcination temperature from the histogram. The pyrolysis of the sample can be accomplished inside the AFM chamber under a flow of dry air.

### Reactor System for Ethylene Epoxidation

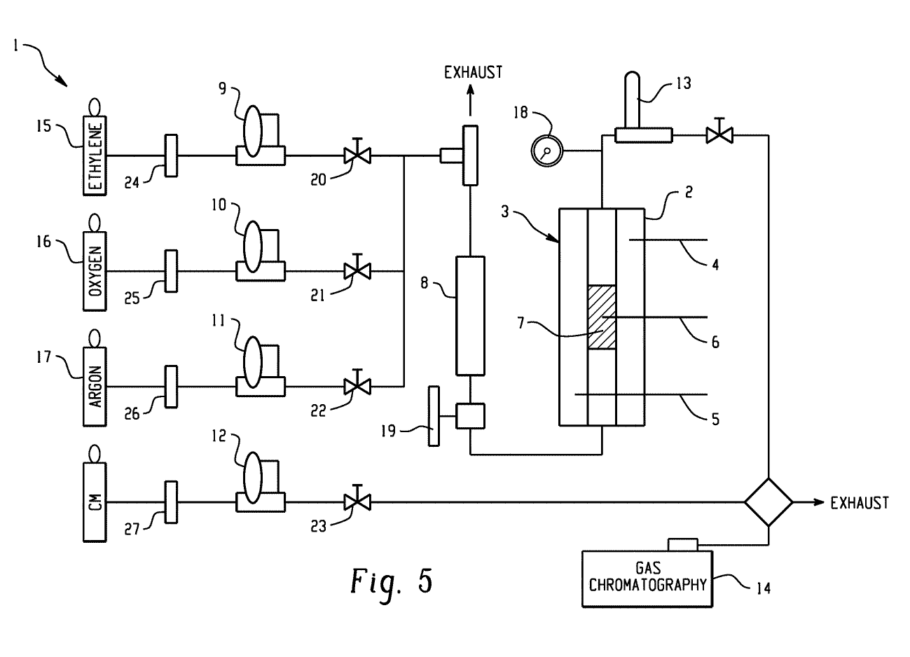

The reactor system for oxidizing ethylene to ethylene oxide in the presence of a supported gold nanoparticle catalyst can be of any kind suitable to provide sufficient contact between gas, liquid and solid phase, such as fixed bed flow reactors, bubble column reactors, slurry-stirred tank reactors with fixed or distributed ethylene-injection and the like. In one embodiment, the selective oxidation of ethylene to ethylene oxide is performed in a fixed bed flow reactor. In one embodiment, oxidizing ethylene to ethylene oxide is carried out in a fixed bed flow reactor. FIG. 5 represents an exemplary reactor system 1 for oxidizing ethylene to ethylene oxide by using the supported gold nanoparticle catalyst of the presently disclosed subject matter.

In one non-limiting example, as shown in FIG. 5, a stainless steel fixed bed reactor tube 2 with internal diameter of about 1 cm is packed with the supported gold nanoparticle catalyst of the presently disclosed subject matter with a fixed bed length of about 10 cm. The reactor tube 2 is covered with a three heating zone furnace 3 equipped with thermocouples 4 and 5 to measure the temperature in each section. Another thermocouple 6 is placed inside the reactor tube 2 at the center of the catalyst bed 7. The gaseous reactants of the reaction mixture include ethylene 15 and oxygen 16. Additionally, in some embodiments, the gaseous mixture includes an inert gas 17. In one embodiment, the inert gas is argon. The inert gas acts as a heat remover as the reaction is exothermic. The inert gas does not affect the catalyst properties. The gaseous reactants are admixed prior to being introduced into the reactor system. The gaseous reactant mixture is pre-heated to a temperature of from about 100° C. to about 180° C. at a pre-heater 8 before it goes into the reactor tube 2. Additionally and alternatively, the gaseous reactants can be pre-heated individually. The reactor system 1 is equipped with mass flow controllers 9, 10, 11 and 12 for all gaseous reactants to monitor the flow rate of the gases before they go to the pre-heater 8. The reactor system 1 includes a back pressure regulator 13 connected after the outlet of the reactor tube 2 to control the reaction pressure. The reaction pressure is initially provided by the feed of the gaseous reactants and after the reaction has commenced, is maintained by the use of the back-pressure regulator 13. The reaction pressure can be from about 10 bar to about 25 bar. In some embodiments, the reaction pressure is from about 15 bar to about 22 bar. As shown in FIG. 5, the reactor tube 2 also includes a pressure gage 18, a rupture disk 19, four check valves 20-23, and four filters 24-27.

All the tubes connecting the reactor to a gas chromatograph 14 is covered with heating tape that can be heated to 130° C. in order to prevent any condensation of the products in the tubing. Gas chromatography (GC) analysis of the reactant feed and the product gases is performed in a Hewlett-Packard 6890 instrument equipped with a flame ionization detector (FID) and a thermal conductivity detector (TCD). The GC oven temperature and the injection port, which can be equipped with 1 ml sample loop, are operated at 150° C. A suitable calibration gas mixture including ethylene oxide, ethylene, nitrogen, oxygen, carbon monoxide, argon, and carbon dioxide is used for calibration of the GC and for determining the response factors for each gas. For other expected liquid products, such as acetaldehyde, acetic acid, and formaldehyde, standard solutions can be prepared for the GC calibration.

The reaction temperature is provided by placing the catalyst bed within the reaction tube having walls placed in a furnace heated to the desired reaction temperature. The reaction temperature for oxidizing ethylene to ethylene oxide can be from about 160° C. to about 220° C. In some embodiments, the reaction temperature is from about 180° C. to about 200° C.

The oxygen concentration in the feed gas mixture can vary widely, from about 0.1% to about 50% or higher of the feed mixture by applying proper measures to avoid explosion problems. The oxygen can come from air or pure oxygen source. In one embodiment, air is the source of oxygen in the feed.

Ethylene conversion and product selectivity can be calculated according to the following equations 1 and 2:

$$\begin{matrix}Ethylene conversion ( \% ) = \frac{( Ceth , in ) - ( Ceth , out )}{( Ceth , in )} \times 100 & eq . 1 \\ Product selectivity ( \% ) = \frac{( Cj )}{( Cj , in ) - ( Cj , out )} \times 100 & eq . 2\end{matrix}$$

Where Cⱼ is the molar fraction of the component j, the yield is the results of multiplying of the conversion and the selectivity, equation 3:

$$Product yield ( \% ) = \frac{( Cj )}{( Ceth , in )} \times 100 \qquad eq . 3$$

### Effect of Preparation Parameters

Various preparation parameters including the type and/or nature of the support, the gold precursor, the pH value, the gold concentration, and the alkali promoters can impact the catalytic activity and selectivity of the supported gold nanoparticle catalysts. At a standard set of reaction conditions and gold concentrations, the effect of the preparation parameters can be evaluated in four stages. In the first stage, the optimum pH value for the precipitation of the support hydroxide from metal salts is determined. In one embodiment, the pH value is from about 5 to about 10. This can be accomplished for all the supports before supporting a specific tri-alkyl-phosphine gold (I) nitrate complex. The pH value can be adjusted by using a 10% potassium hydroxide solution. In the second stage, the effect of the support and the nature of the chemical interaction between the support and the gold complex are studied. In this stage, all gold complexes are supported over each oxide support at a fixed gold load and are evaluated at fixed reaction conditions. The third stage identifies the appropriate gold loading. In one embodiment, the supported gold nanoparticle catalyst of the presently disclosed subject matter includes about 0.1% wt % to about 5 wt % (e.g., from about 0.2 wt % to about 5 wt %) of nitro (phosphorus compound) gold (I) complex (e.g., nitro (tri-alkyl phosphine) gold (I) complex). In the fourth stage, the effect of the alkali promoter on the catalytic activity and selectivity of the supported gold nanoparticle catalyst are determined. In one embodiment, the alkali promoter is cesium. Cesium nitrate can be introduced with different concentrations to the solutions of the gold complexes before addition to the support.

### Mechanism and Kinetics

The supported gold nanoparticle catalysts disclosed herein exhibit superior catalytic activity and selectivity for ethylene epoxidation, and can be used for studying the reaction kinetics and mechanism. All the kinetics analysis can be performed using a fixed gold load, fixed bed length, and fixed reaction pressure. The ethylene and oxygen conversion can be maintained at 10% level or less. Several sets of analysis can be performed to investigate the reaction mechanism. The first is to study the effect of the reactant partial pressure on the reaction rate at fixed reaction conditions. The concentration of each reactant can be varied in the range of from about 1% to about 15% while the concentration of the other reactant can be maintained at about 15%. The total flow rate can be adjusted by the flow of argon gas. This can eventually facilitate the determination of the rate constant k and the order of the reaction with respect to each reactant and the overall order of reaction.

A second set of analysis is to evaluate the influence of the contact time of gases over the catalyst bed on the reaction rate and catalyst selectivity. This can be attained by a variation of the total flow rate in the range of from about 50 ml/min to about 300 ml/min at a fixed reactant composition and fixed reaction conditions.

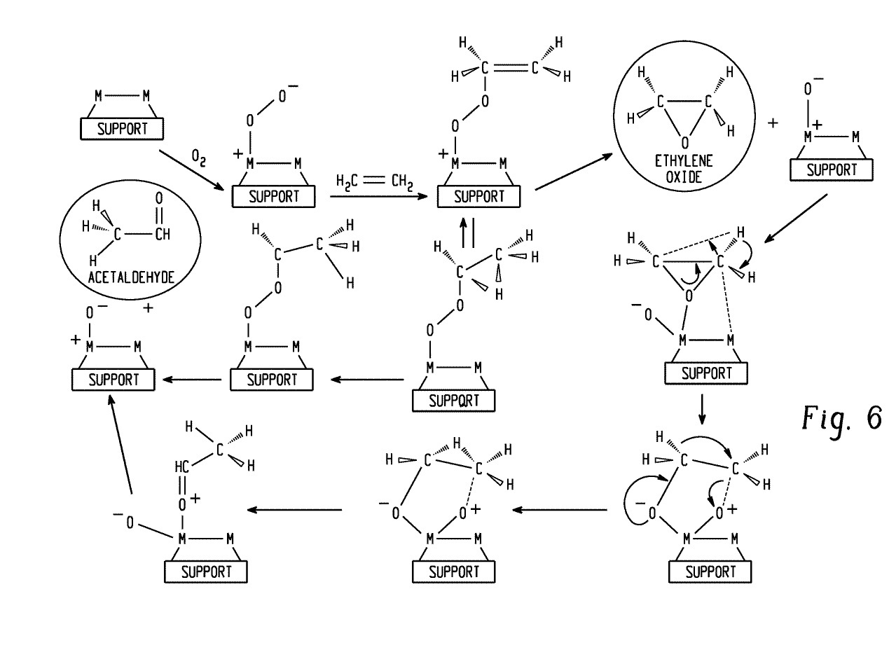

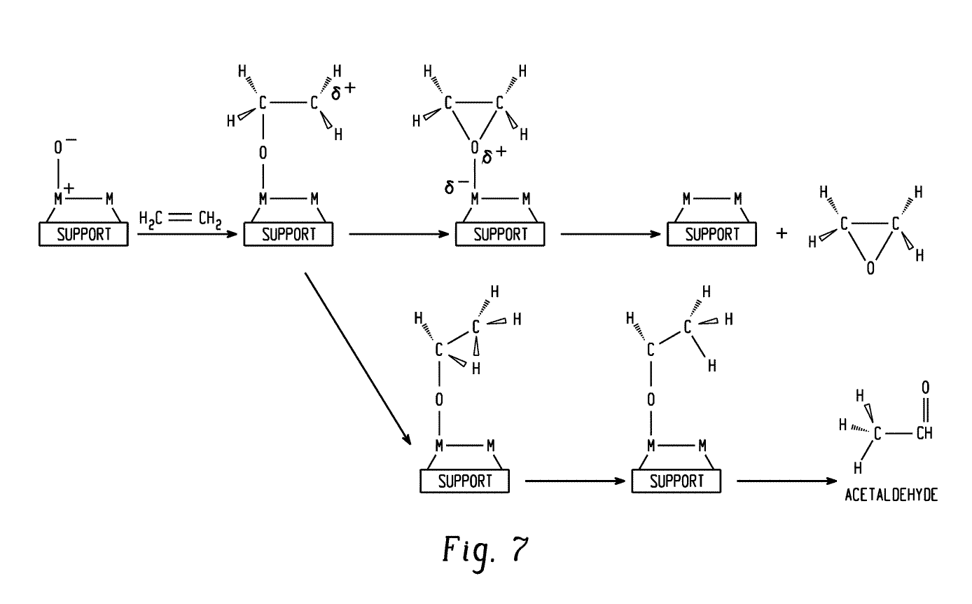

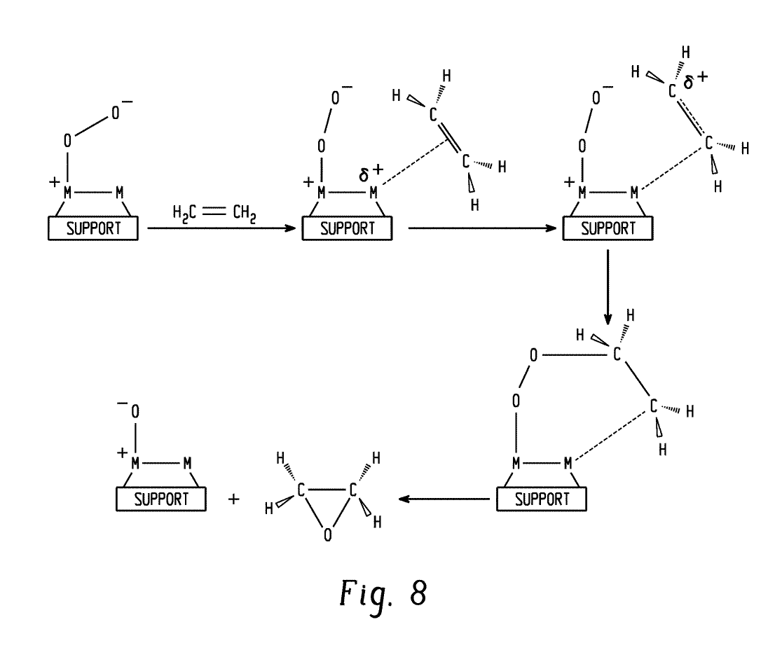

The third set of analysis is to examine the effect of the reaction temperature in the range of from about 50° C. to 300° C. on the reaction rate and ethylene conversion. Additionally, the apparent activation energy can be calculated directly from the Arrhenius equitation. FIG. 6 represents one mechanism for the selective oxidation of ethylene to ethylene oxide over supported gold nanoparticle catalyst synthesized by the process of the presently disclosed subject matter. Another mechanism suggests that either atomic oxygen chemisorbed on the surface is the active phase for the selective epoxidation of ethylene, or ethylene is chemisorbed on the surface with oxygen and reacts over the surface according to Langmuir-Hinshelwood mechanism, as shown in FIGS. 7A and 7B.

The term “about” or “substantially” means within an acceptable error range for the particular value as determined by one of ordinary skill in the art, which will depend in part on how the value is measured or determined, i.e., the limitations of the measurement system. For example, “about” can mean a range of up to 20%, up to 10%, up to 5%, and or up to 1% of a given value.

As used herein, “alkyl” refers to a saturated or unsaturated hydrocarbon including 1-20 carbon atoms including both acyclic and cyclic structures (such as cyclohexane and the like). Exemplary alkyls include, but are not limited to, methyl, ethyl, propyl, i-propyl, isopropyl, butyl, t-butyl, iso-butyl, sec-butyl, pentyl, hexyl, cyclopropyl, cyclobutyl, cyclopentyl, cyclohexyl, propenyl, butenyl, and cyclohexenyl. A linking divalent alkyl group is referred to as an “alkylene,” including, but not limited to, ethylene, and propylene.

As used herein, the term “aryl” refers to monocyclic or polycyclic (e.g., having 2, 3 or 4 fused rings) aromatic hydrocarbons. Exemplary aryls include, but are not limited to, phenyl, naphthyl, anthracenyl, phenanthrenyl, indanyl, and indenyl. In some embodiments, aryl groups have from 6 to about 20 carbon atoms, from 6 to about 15 carbon atoms, or from 6 to about 10 carbon atoms.

As used herein, the term “aralkyl” refers to alkyl substituted by aryl. One example of aralkyl is benzyl.

In accordance with the presently disclosed subject matter, all of the above-described “alkyls,” “aryls,” and “aralkyls” can be optionally substituted. As used herein, the term “substituted” means that a group be further substituted with one or more groups selected from oxygen, nitrogen, sulphur, alkyl, alkenyl, alkynyl, aryl, halo, haloalkyl, haloalkenyl, haloalkynyl, haloaryl, hydroxy, alkoxy, alkenyloxy, alkynyloxy, aryloxy, carboxy, benzyloxy, haloalkoxy, haloalkenyloxy, haloalkynyloxy, haloaryloxy, nitro, nitroalkyl, nitroalkenyl, nitroalkynyl, nitroaryl, nitroheterocyclyl, azido, amino, alkylamino, alkenylamino, alkynylamino, arylamino, benzylamino, acyl, alkenylacyl, alkynylacyl, arylacyl, acylamino, acyloxy, aldehydo, alkylsulphonyl, aryisulphonyl, alkylsulphonylamino, arylsulphonylamino, alkylsulphonyloxy, arylsulphonyloxy, heterocyclyl, heterocycloxy, helerocyclylamino, haloheterocyclyl, alkylsulphenyl, arylsulphenyl, carboalkoxy, carboaryloxy, mercapto, alkylthio, arylthio, acylthio, and the like, having from 1 to 20 carbon atoms.

Although the presently disclosed subject matter and its advantages have been described in detail, it should be understood that various changes, substitutions and alterations can be made herein without departing from the spirit and scope of the presently disclosed subject matter as defined by the appended claims. Moreover, the scope of the presently disclosed subject matter is not intended to be limited to the particular embodiments of the process, machine, manufacture, and composition of matter, means, methods, and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such modifications.

1. A process for preparing a supported gold nanoparticle catalyst, the process comprising:
adding a solution of a phosphorus compound to a solution of chloro (dimethyl sulfide) gold (I) to obtain a solution of chloro (phosphorus compound) gold (I) complex,
wherein the phosphorus compound is selected from the group consisting of a phosphine having a formula of PR₁R₂R₃, a phosphinite having a formula of P(OR₄)R₅R₆, a phosphonite having a formula of P(OR₇)(OR₈)R₉, a phosphite having a formula of P(OR₁₀)(OR₁₁)(OR₁₂), or a combination comprising at least one of the foregoing; and
wherein R₁ to R₁₂ are each independently an alkyl, substituted alkyl, aryl, substituted aryl, aralkyl, substituted aralkyl, or a combination comprising at least one of the foregoing;
adding the solution of chloro (phosphorus compound) gold (I) complex to a solution of silver nitrate to obtain a solution of nitro (phosphorus compound) gold (I) complex;
applying the solution of nitro (phosphorus compound) gold (I) complex to a metal hydroxide support;
drying the metal hydroxide support; and
calcining the dried metal hydroxide support to form the supported gold nanoparticle catalyst.

2. The process of claim 1, wherein the metal hydroxide is aluminum hydroxide, magnesium hydroxide, zinc hydroxide, iron hydroxide, nickel hydroxide, titanium hydroxide, or a combination comprising at least one of the foregoing.

3. The process of claim 1, wherein the metal hydroxide is obtained by hydrolysis of the metal in a solution of potassium hydroxide.

4. The process of claim 2, wherein the metal hydroxide is titanium hydroxide.

5. The process of claim 4, wherein the titanium hydroxide is obtained by hydrolysis of titanium-tetra-isopropoxide in a solution of potassium hydroxide.

6. The process of claim 3, wherein the hydrolysis is carried out in a pH value of from about 5 to about 10.

7. The process of claim 6, wherein the pH value is adjusted by addition of the potassium hydroxide solution.

8. The process of claim 1, wherein the solution of nitro (phosphorus compound) gold (I) complex is applied to the metal hydroxide with a continuous stirring for about 12 hours.

9. The process of claim 1, wherein the metal hydroxide is dried under vacuum at a temperate of from about 20° C. to about 25° C.

10. The process of claim 1, wherein the dried metal hydroxide is calcined at a temperate of from about 100° C. to about 300° C.

11. The process of claim 1, wherein the process is carried out in an inert nitrogen atmosphere.

12. The process of claim 1, further comprising adding an alkali promoter to the solution of nitro (phosphorus compound) gold (I) complex before applying the solution of nitro (phosphorus compound) gold (I) complex to the metal hydroxide support.

13. The process of claim 12, wherein the alkali promoter is cesium.

14. The process of claim 1, wherein the phosphorus compound is the phosphine of formula PR₁R₂R₃.

15. The process of claim 1, wherein
the alkyl is i-propyl, cyclohexyl, t-butyl, ethyl, or a combination comprising at least one of the foregoing;
the aryl is phenyl;
the substituted aryl is mesityl, 2,6-dimethoxyphenyl, 4-methoxyphenyl, 1-naphthyl, 4-methylphenyl, or a combination comprising at least one of the foregoing; and
the aralkyl is benzyl.

16. A supported gold nanoparticle catalyst prepared by the process of claim 1.

17. The supported gold nanoparticle catalyst of claim 16, wherein the size of the gold nanoparticle is from about 2 nm to about 15 nm.

18. The supported gold nanoparticle catalyst of claim comprising from about 0.2% to about 5% by weight of the nitro (phosphorus compound) gold (I) complex.

19. A process for oxidizing ethylene to ethylene oxide, comprising reacting ethylene and oxygen in the presence of the supported gold nanoparticle catalyst of claim 16.

20. The process of claim 19, wherein the process is carried out in a fixed bed flow reactor, optionally wherein an inert gas is fed to the oxidization process.
